(19) United States
(12) Patent Application Publication
(10) Pub. No.: US 2010/0267168 A1
(43) Pub. Date: Oct. 21, 2010

(54) METHOD FOR END TITRE DETERMINATION AND THE EVALUATION THEREOF BY MEANS OF AN INDIRECT IMMUNOFLURESCENCE ASSAY

(75) Inventor: Rico Hiemann, Thiendorf Ot Sacka (DE)

Correspondence Address: URSULA B. DAY, ESQ., 708 Third Avenue, SUITE 1501, NEW YORK, NY, 10017, US

(73) Assignee: Medipan GmbH

(21) Appl. No.: 12/741,341

(22) PCT Filed: Nov. 13, 2008
(86) PCT No.: PCT/DE08/01894

§ 371 Date: May 4, 2010

(30) Foreign Application Priority Data
Nov. 13, 2007 (DE) 10 2007 054 792.9

(51) Int. Cl. G01N 33/543; G01N 30/96

(52) U.S. Cl. 436/518; 422/69; 422/61

(57) ABSTRACT

The invention relates to a method for end-titre determination in the determination of antibodies against nuclear and cytoplasmic antigens in human sera by means of an indirect immunofluorescence assay. The invention further relates to a kit for in vitro diagnosis for determining antibodies against nuclear and cytoplasmic antigens in human sera by means of an indirect immunofluorescence assay and a computer program for evaluation and for determination of the end titre within the framework of said method.

The invention relates to a method for end titre determination in the determination of antibodies against nuclear and cytoplasmic antigens in human sera by means of an indirect immunofluorescence assay. The invention further relates to a kit for in vitro diagnosis for determining antibodies against nuclear and cytoplasmic antigens in human sera by means of an indirect immunofluorescence assay and a computer program for evaluation and for determination of the end titre within the framework of said method.

### BACKGROUND OF THE INVENTION

Autoimmune diseases are diseases caused by an over-reaction of the immune system against the body's own tissue. The immune system mistakenly detects the body's own tissue as foreign matter to be attacked. Through this, serious inflammatory reactions arise that can lead to damage of the affected organs. T-cells are responsible for the detection of foreign matter. T-cells are trained in the thymus to dock only on MHC-molecules and to tolerate the body's own matter. In autoimmune diseases these cells behave against their nature. Instead of defending against penetrating foreign matter, they attack the body's own structures. Organs and tissues that are essential for the life of the organism are recognized by the immune system as foreign. The immune system directs its entire strength against these structures including cellular and humoral defence reactions, which result in autoantibodies being generated. These organs and tissues therefore lose their function over time. The invention is therefore directed towards the diagnosis and later treatment of autoimmune diseases.

In principle a serological characterisation of autoimmune diseases is possible through the detection of autoantibody profiles. The majority of these antibodies are directed against nuclear and cytoplasm antigens. Anti-nuclear antibodies (ANAs) are predominantly associated with rheumatic disorders. Some of these ANAs are disease-specific and are used as diagnostic markers. Such antibodies include for example antibodies against:
Double-stranded DNA (ds-DNA) and the Sm-antigen in systemic lupus erythematosus (SLE)
Fibrillarin in scleroderma, topoisomerase I (Scl-70) in diffuse scleroderma, centromeres (ACA) in CREST disease
Histidyl-tRNA-Synthetase (Jo-1) in polymyositis
PM-Scl in the overlap between polymyositis and scleroderma.

ANAs with varied prevalence are found in several disorders. These include: anti-histone antibodies in SLE, in medication-induced Lupus and in chronic nutritive toxic liver disease: anti-RNP-antibodies in SLE and Sharp Syndrome (MCTD: mixed connective tissue disease), and anti-SS-A (Ro) and anti-SS-B (La) antibodies in SLE and Sjögren's syndrome. Anti-mitochondrial antibodies (AMA) of the anti-M2 type react with proteins of the alpha keto acid dehydrogenase complexes of the mitochondria and are characteristic markers for primary biliary cirrhosis (PBC), a chronic cholestatic liver disease.

The earliest method for the detection of ANAs and AMAs is the immunofluorescence test (IFT), whereby frozen tissue sections or single cells are used as a substrate. In this method the different species specificity of the antibody to be detected is a fundamental criterion. For some human antibodies it could be shown that they react exclusively with tissue from humans or primates, whereas other autoantibodies react in a species-unspecific manner to tissue sections from rat, mouse, rabbit or guinea pig. The respective antigens differ in respect to their phylogenetic development. The more species-unspecific antigens remain more strongly conserved in the course of evolution and are therefore found in more distantly related species. The disadvantage that not all animal cells are suited for the detection of specific autoantibodies can however be ignored when using HEp-2 cells.

The so-called HEp-2 cells refer to a human larynx epithelial cell line that exhibits a high specificity for most human autoantibodies directed against nuclear antigens (ANA/ENA) (Hollingworth et al., Clin. Diagn. Lab. Immunol. Vol. 3, 1996 374-377). Systemic rheumatic inflammatory diseases, for example systemic lupus erythematosus (SLE) and variations thereof, progressive systemic sclerosis (PSS), primary Sjögren's syndrome, dermatomyositis, Sharp syndrome (mixed connective tissue disease—MCTD) or rheumatoid arthritis (RA) are characterised by the appearance of a number of autoantibodies directed against components of the cell nucleus and cytoplasm. Although the aethiopathogenetic role of these autoantibodies has not been fully elucidated, they can be applied as markers for various clinical profiles in addition to activity parameters (Tan E. M., Adv Immunol 1982, 33:167-240; Tan E. M., Adv Immunol. 1989, 44: 93-151).

A suitable method in autoantibody diagnostics at the present time is the so-called indirect immunofluorescence test. The immunofluorescence test on HEp-2 cells is a sensitive screening assay for the determination of anti-nuclear antibodies (ANA), that in addition to the recognition of fluorescence patterns, provides evidence for specific underlying antigens and associated disorders (Moore et al., Cancer Res. 1955, 15: 998-602; Weller et al., Proc. Soc. Exp. Biol. Med. 1954, 86: 789-794). In the indirect immunofluorescence assay, HEp-2 cells of a human epithelial cell line are used as a substrate, which have a high sensitivity for most human autoantibodies directed against nuclear antigens (ANA/ENA). HEp-2 cells (human epithelial cells) are provided at this time by various manufacturers (for example INOVA Diagnostics, San Diego, USA; Kallestadt, Chaska, USA; Immuno Concepts, Sacramento, USA).

An indirect ANA HEp-2 immunofluorescence assay for qualitative and semi-quantitative ANA determination proceeds as follows: The antibodies in diluted patient samples and controls react in the first reaction step specifically with the antigens of the HEp-2 cells which are fixed to a slide. Unbound components are removed by a wash step after a 30-minute incubation at room temperature. The bound antibodies react in a second reaction step specifically with anti-human antibodies (IgG and light chain specific), which are coupled to fluorescein isothiocyanate (FITC). Excess conjugate molecules are removed from the immune complex, which is bound to the solid phase, by a further wash step after a 30-minute incubation at room temperature. After being covered the slide is manually read under a fluorescence microscope (excitation wavelength 490 nm, emission wavelength 520 nm). Specific fluorescent patterns are detected according to the histological arrangement of the antigens in the HEp-2 cells.

One of the most significant problems with the indirect immunofluorescence assay is the evaluation of fluorescent optical images in autoantibody diagnostics on HEp-2 cells. According to the present state of the art in autoantibody diagnostics an automated method using HEp-2 cells exhibits essentially the following features: A system for image acquisition by means of a fluorescence microscope and digital camera, comprising an automatic image analysis and determination of the describing features of fluorescent patterns, an automatic classification of fluorescent patterns and output of the recognised pattern on a laboratory data system. For example, a fluorescent microscope with a camera and a standard PC serves as an acquisition unit. The fluorescent pattern that results from the measurement enables the recognition of seven different basic patterns at the present time (homogenous, nucleolar, finely speckled, coarsely speckled, centromeric, peripheral, multiple nuclear points).

Such a system is for example known from DE 198 01 400 C1. DE 198 01 400 C1 describes a method and system for the automatic recognition, property-description and interpretation of HEp-2 cell patterns. This method and the corresponding system serve to detect autoimmune diseases, whereby the interpretation of HEp-2 cells takes place over a two-dimensional image capture and digitalisation, distribution of the sectioned HEp-2 cells in the background of the image, classification in a number of discrete image-classes, summing of pixels into individual objects, determination of the features of the objects, comparison of the cell patterns and display and/or saving of the cell patterns and the assigned class affiliation. The system according to DE 198 01 400 C1 consists of a recording device and an image-segmenting device, a class-image classifying device, a feature-characterizing device and a cell pattern-comparing device. The devices are contained and linked one after the other in a data-processing computer. A similar system is also described in EP 1 733 333 B1.

The aforementioned methods are based on the principle of end point titration, which is a semi-quantitative method for the determination of the amount of an antibody in a serum. In this method a serial dilution of the serum, and therefore antibody, is tested with a constant volume. The result is indicated as the reciprocal value of the highest dilution factor in which an immunofluorescent pattern was still visible. The problem with this approach is that a qualitative positive test result alone does not provide or allow a substantiated diagnostic statement. Only semi-quantitative determination, which means titration of sera with the specification of the end point titre, leads to a diagnostically relevant statement.

However, it must be said that a clinical diagnosis is not unproblematic. This is because up to 10% of the general population could exhibit ANAs. The appearance of ANAs is dependent on the age and gender of the patient, whereby the frequency increases with increasing age. A positive result with a low titre without clinical symptoms can therefore be seen as normal for elderly persons. Healthy young people are therefore usually ANA negative. SLE patients undergoing corticosteroid therapy can also be ANA negative. ANA can also appear in relatives of patients with connective tissue diseases, who could also later become ill.

At the present time the evaluation of fluorescent patterns, based on the different fluorescence intensity of individual fluorescent objects, is classified according to the following recommendation from the Center for Disease Control and Prevention (CDC), Atlanta, USA (Lyerla and Forrester: The Immunofluorescence (IF) test. In: Immunofluorescence methods in virology, USDHHS, Georgia, 1979, 71-81):

4+=maximum fluorescence, brilliant yellow-green
3+=less brilliant yellow-green fluorescence
2+=clear, but mat yellow-green fluorescence
1+=very weak suppressed fluorescence

The intensity of the fluorescence does however not reflect the antibody concentration. Differences in opticals, filters and light sources in various microscopes can lead to differences in the fluorescence intensity of more than one step. In this respect it comes down to so-called “negative” and “positive” results. A sample dilution is assessed as “ANA negative” when the HEp-2 cells exhibit fluorescence smaller than 1+ and the absence of a determinable pattern. A sample dilution is assessed as “ANA positive” when the HEp-2 cells exhibit a fluorescence of 1+ or more in addition to a clearly determinable pattern.

The determination of the titration end point is therefore also dependent on the type and condition of the fluorescent microscope, on the enlargement of the objective in addition to the subjective judgement of the observer. Samples or wash buffer solutions contaminated with bacteria could lead to unspecific colouring of the cell substrate.

In a semi-quantitative titration, the last dilution factor in which a 1+ fluorescence signal is present is given as the result. This titration factor is then given as the end point titre for the serum. The titre is the reciprocal value of the dilution factor. In the four-fold dilution series recommended by the Center for Disease Control and Prevention (CDC), the end point of the titration can be extrapolated as the following:

1:40=3+
1:160=2+
1:640=+/−
1:2560=−
The extrapolated titre is thus 1:320.

As a result of this evaluation, titres of 40 and 80 are considered as low positives, but clinically irrelevant, 160 and 320 are considered as moderate titres which could be clinically relevant, whereas titres of 640 or more are considered as highly positive and clinically relevant. Producing dilution series of this type and carrying out the respective tests is however time consuming and also very cost intensive; therefore in practice it is common that the four-fold dilution series is waived and the analysis is limited to one or two dilutions (for example 1:80 and 1:320).

For example, the following system is used in the prior art for carrying out the titre determination: The applied system contains the preparation to be investigated, in addition to the use of a motorised X-Y-sample table, a motorised fluorescence microscope (with Z-control) including a controllable camera and lastly a personal computer with corresponding software and access to a databank.
Day 1: Entry screening with 1:80 (+1:320); dilution with visual estimation of end titre by a medical technical assistant (MTA);
Day 2: Dilutions of 1:640, 1:1280 and optionally further dilution steps up to 1 over and under the estimated end titre.

If the end titre could not be determined, then further dilutions are necessary until the fluorescent pattern is no longer visible.

The striking problem with this method is that the fluorescence intensity of the preparation is dependent on many factors, for example the sensitivity of the specific camera that has been applied, the staining protocol, the anti-bleaching medium, the excitation light, which in turn is dependent on the age of the light source and the applied optical components such as the objective and set of filters.

The disadvantages of the methods used in the current state of the art can be summarised by the following:
The evaluation of fluorescent patterns still occurs in a purely subjective manner by the observer.
The intensity of the fluorescent pattern is subject to large fluctuations, as the patterns are dependent on many technical parameters, for example the sensitivity of the specific camera that has been applied, the staining protocol, the anti-bleaching medium, the excitation light, which in turn is dependent on the age of the light source and the optical components used in the optical analysis. Finally, the intensity and “brightness” of a fluorescent pattern is perceived differently by every observer. The subjective influence on the “estimation of brightness” should therefore not be ignored.
The evaluation itself is limited to the capabilities of the laboratory and for the most part the minimum that is deemed necessary. This means that only incomplete measurements or dilution series are carried out and a titration end point is then extrapolated which is more or less exact according to the experience of the respective user. This end point can therefore lie above or below the actual end titre. “Correct” results often remain left to chance.
The subjective evaluation of fluorescent patterns through phenotypic features leads to an increased error rate and thereby even to a mistaken interpretation of the images to be evaluated. In diagnostic practice this can lead to either autoimmune diseases not being recognised as such, or false positive results being determined and evaluated. In diagnostic and clinical practice this could lead to possible omission of, or premature, treatment, whereby both scenarios represent a more than unsatisfactory situation for the patient.

### DETAILED DESCRIPTION OF THE INVENTION

The technical problem to be solved in light of the prior art is to provide an objective and reproducible evaluation of fluorescence patterns in autoantibody diagnostics by means of an indirect immunofluorescence assay.

This problem is solved through the features of the independent claims, in addition to the respective dependent claims.

According to the present invention a method for end titre determination in the determination of antibodies against nuclear and cytoplasmic antigens in human sera by means of an indirect immunofluorescence assay is intended, comprising multiple method steps, whereby a reaction and binding of autoantibodies contained within patient sera occurs with and to antigens from HEp-2 cells, leukocytes, Crithidia luciliae, and/or tissue sections which are fixed to a slide. A specific fluorescent marking of the bound autoantigens takes place, followed by a fluorescent microscopic analysis of the fluorescently marked autoantibodies bound to the slide, in addition to optical recording and evaluation of the fluorescent optical images using the fluorescence intensity in an evaluation system. The latter evaluation system is calibrated before the binding of autoantibodies contained within patient serum with and to antigens from HEp-2 cells, leukocytes, Crithidia luciliae, and/or tissue sections which are fixed to a slide, by means of at least two control sera with defined titre for the creation of a dilution series, whereby the intensity of the excitation light is also measured. The fluorescence intensity of the recorded fluorescent optical images is set in relation to the titre of the control sera, thereby providing the end titre of the patient serum to be investigated. The end titre of the patient serum to be investigated arises from the determined reference function of the calibrated system, which, depending on the pattern or pattern combination, can be linear or non-linear.

The method according to the present invention for end titre determination is characterized by a calibration phase, in addition to measurement of the intensity of the excitation light. Furthermore, the maximum meaningful exposure time (the final exposure time), in addition to the initial titre of the patient serum and the exposure time of the camera are relevant.

A fundamental feature of the invention is the calibration procedure of the optics, which is used to evaluate the fluorescent patterns. This is achieved through measurement of the excitation light of the fluorescence, and with a slide according to the present invention, exhibiting on its surface multiple control sera with defined titres, by which the optical system is calibrated. For the calibration a measurement of the average exposure time over multiple images is carried out for every defined control serum and subsequently the end point (saturation point) of the camera is determined, which in turn results from the maximum meaningful exposure time, which corresponds to the exposure time of the end titre of the control serum. The end titre of the patient serum to be investigated results therefore from the final exposure time, the initial titre of the patient serum and the exposure time of the camera, preferably according to the determined calibration function which in the simplest linear case can be calculated according to the following equation

$$serum titre = \frac{input titre}{exposure time} * final exposure time$$

Furthermore, a kit for in vitro diagnosis for the determination of antibodies directed against nuclear and cytoplasmic antigens in human serum by means of an indirect immunofluorescence assay is subject matter of the invention, comprising of at least
a. a slide with multiple application sites coated with HEp-2 cells for the application of control and patient sera,
b. control sera with different, pre-defined titres for calibration of the optical system of a immunofluorescence microscopic measurement and evaluation system.
c. fluorescently marked anti-human antibodies for the specific coupling of antibodies that are bound to HEp-2 cells.

The anti-human antibodies to be applied in the kit are anti-human-immunoglobulin and can be optionally coupled to fluorescein-isothiocyanate.

A kit is herein described, which is suitable for carrying out the method according to the present invention for end titre determination in the determination of antibodies directed against nuclear and cytoplasmic antigens in human sera. Advantageously the kit comprises additionally reagents, wash solutions and other solutions, which are tailored for their intended execution. The kit preferably also provides a protocol for every necessary step in the in vitro diagnosis, in addition to optionally provided reference value tables and calibration information. The kit further contains information on combining the contents of the kit.

The invention also provides an immunofluorescence assay on HEp-2 cells as a sensitive screening assay for the determination of anti-nuclear anti-bodies (ANA), which allows a statement about the underlying antigens and associated disorders via the recognition of fluorescence patterns. The kit encompasses a set of reagents for the qualitative and semi-quantitative determination of antibodies directed against antigens in the cell-nucleus and in the cytoplasm of HEp-2 cells in human serum by means of an automated evaluation.

Furthermore, the invention provides a computer programme, which is saved in a computer-readable-medium containing computer-readable-data (programme code) through which the computer is instructed, during active computer operation, to carry out a method according to the present invention. Through the computer programme a method for end titre determination in the determination of antibodies against nuclear and cytoplasmic antigens in human sera by means of an indirect immunofluorescence assay through the evaluation of fluorescent optical images in autoantibody diagnostics can be electronically controlled and evaluated.

The invention also encompasses a device according to the present invention for end titre determination in autoantibody diagnostics in the determination of antibodies directed against nuclear and cytoplasmic antigens in human sera by means of an indirect immunofluorescence assay, comprising
a. a system for image capture by means of a fluorescence microscope with a camera
b. a system for automatic image analysis and determination of the captured fluorescence patterns and fluorescence intensities of bound autoantibodies from patient serum, which react with and are bound to antigens of the HEp-2 cells, leukocytes, Crithidia luciliae and tissue sections, which are fixed to the slide.

This device has the advantage that it provides both image capture and simultaneous automatic image analysis. This particularly reduces the known disadvantages of the prior art regarding the insufficient analysis of data.

The method according to the present invention and the kit based upon said method are suitable for cell-based assays, in which the patterns, and potentially titres, are automatically read. The method is suited for the qualitative and semi-quantitative determination of antibodies in human serum against antigens in the nucleus and cytoplasm of HEp-2 cells by means of an automatic evaluation. Alongside HEp-2 cells, leukocytes, Crithidia luciliae and tissue sections are also preferred.

The fluorescent signal is subject to variations, which is why the addition of anti-bleaching reagents, for example 2,3,5,6 Tetramethyl-1,4-Phenylenediamine (C₁₀H₁₆N₂), has proven to be advantageous.

A fundamental element of the invention, especially of the kit and underlying method, is that the unit to be investigated “cell+conjugate” stays constant. The conjugate (=colouring agent+antibody) is held stable through a substance which prevents the fading (anti-bleaching) of the colouring agent. The substance 2,3,5,6 Tetramethyl-1,4-Phenylenediamine (C₁₀H₁₆N₂) is preferably used. The fundamental technical parameters (fluorescence filter, camera, objective) are also constant. Therefore only the exposure intensity can fluctuate, which is thus measured in predicting the titre (=measurement of the excitation light). The single variable component of the technology is therefore the light source, which also explains why the excitation light of the fluorescence is measured, as fluorescence excitation and fluorescence emission correlate with each other. The antibody concentration (titre) therefore results from the exposure time of the serum image.

The advantages of the present invention are therefore the objective determination of the end titre, in addition to a fast and cost-effective acquisition of the end titre of a serum. It is possible to universally apply the system according to the present invention on various measurement systems. Furthermore, different kinds of preparations can be used (the system can be universally applied for different preparations such as cells, single cells or tissue sections). Additionally, technical and financial resources can be saved through the present invention.

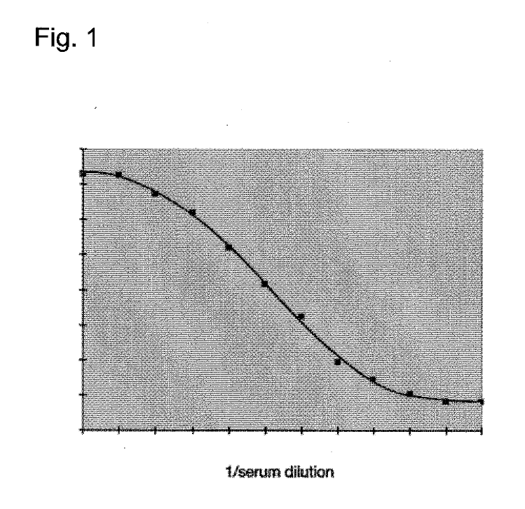

Further advantageous elements are described in the dependent claims. The invention is also more clearly described through the examples and figures, although the invention is not intended to be limited by the examples disclosed herein. FIGS. 1 to 4 demonstrate different calibration curves.

### EXAMPLES

The invention is intended to be more clearly described in light of the figures representing the examples, although the invention is not intended to be limited by the examples disclosed herein.

I. Preparation and execution of the measurements. The following method is applied:
1. The fluorescence optics are calibrated with several control sera.
2. The patient sera are pipetted onto the intended application sites of the coated slide at room temperature and incubated for 30 minutes at room temperature in a humid incubation chamber.
3. lides are rinsed with PBS solution and washed in a staining tray 2× for 5 minutes in fresh PBS.
4. The slides are covered with a conjugate solution and incubated in a humid incubation chamber with UV-light protection for 30 minutes at room temperature.
5. Step 3 is repeated.
6. The slides are covered with a coverslip without bubbles and measured under a fluorescence microscope.

### II. Calibration

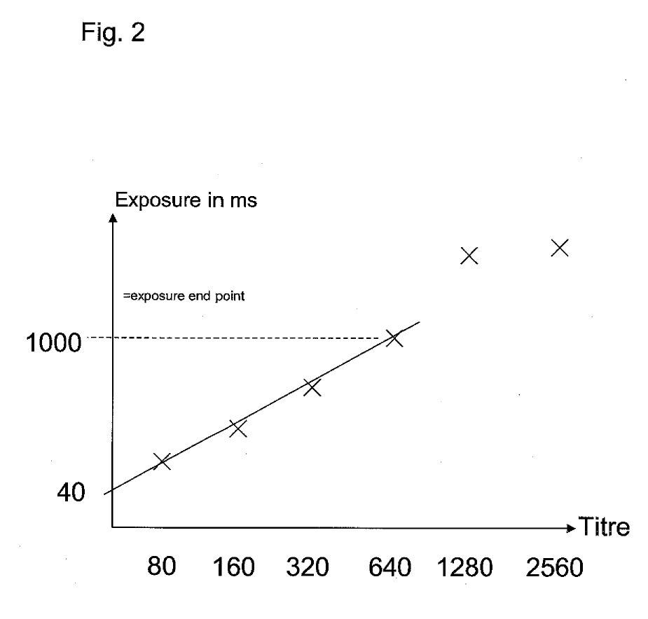

FIG. 2 shows calibration with a first calibration serum 1, whereby the end point is known (640). The regression of the measurements of exposure times for titrated-out serum is in the most simple case a straight line (Y=m×+b), whereby m is the slope and b the intersection with the Y-axis. More complex curves can be calculated according to the following formula:

$$sig ( t ) = \frac{1}{1 + {e}^{- t}}$$

Because the end point of the calibration serum is known, only measurement points up until the end point of the regression should be included, as only autofluorescence of the tissue is measured beyond this point.

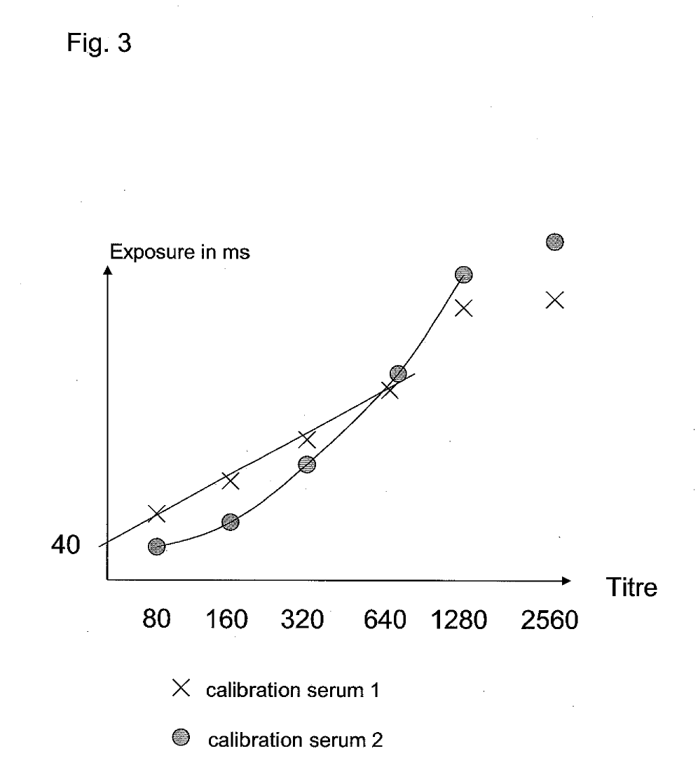

FIG. 3 shows the (homogenous) calibration serum 1 with a known end point (640) in correlation to a second calibration serum 2 (dot pattern) whose end point is also known (1280). Different curve shapes for the various calibration sera could arise, depending on the pattern and specificity (linear slope or exponential or sigmoidal). Every calibration serum has a different antibody specificity (homogenous pattern, centromere pattern, dot pattern, etc. . . . (see below)). In practise during the measurement the pattern is determined, the calibration curve selected and the end titre point of the serum is calculated from the introduction of the dilution titre in the calibration curve.

### III. Titre Estimation of the Serum

An HEp-2 end titre estimation with an initial titre of 1:80 was carried out.

The “calibration slide” was calibrated as follows (see example 1, point 1): A first serum with a known titre of 1:640 was applied in 8 dilutions to 8 wells on the slide and exposed and measured by means of fluorescence microscopy. The fluorescence intensity was measured at different exposure times. This resulted in the following series of measurements:

TABLE 1
Calibration of the slide

| Well | Dilution | Measured exposure time | Comment |
|---|---|---|---|
| 1 | 1:40 | 290 ms | |
| 2 | 1:80 | 602 ms | |
| 3 | 1:160 | 1200 ms | |
| 4 | 1:320 | 2510 ms | |
| 5 | 1:640 | 5000 ms | End point |
| 6 | 1:1280 | 6100 ms | Saturation (autofluorescence |
| | | | of the preparation) |
| 7 | 1:2560 | 6105 ms | |
| 8 | 1:5120 | 6205 ms | |

The true shape of the function was determined by regression from the values of wells 1 to 5 (see FIG. 1; Y-axis: light intensity; X-axis: dilution). In this example the maximum meaningful exposure time was 5000 ms, as afterwards the autofluorescence of the preparation begins and would thereby distort the results. Due to the described calibration of the slide (calibration slide) the end point of the calibrated system in this example is known to be 5000 ms.

The slide that carries the patient sample (patient slide) can exhibit however different dilutions. In this instance the dilution of the serum in the well is known to be 1:80. The images are automatically exposed so that the signal of the immunofluorescence is completely captured by the sensor of the camera (see Hiemann et al. Cytometry Part A 69A (2005)). An average exposure time of the camera of 500 ms is measured; this arises from averaging the exposure times of the single images of the well. In practice the question that remains to be answered is therefore: How high is the expected end titre of the serum?

The solution based on the invention leads to the result that, with a linear relationship, the end titre of interest can be calculated according to the determined reference function of the calibrated system. According to the above example this is carried out by

$$\begin{matrix}serum titre = \frac{input titre}{exposure time} * final exposure time \\ = \frac{80}{500 ms} * 5000 ms \\ = 800\end{matrix}$$

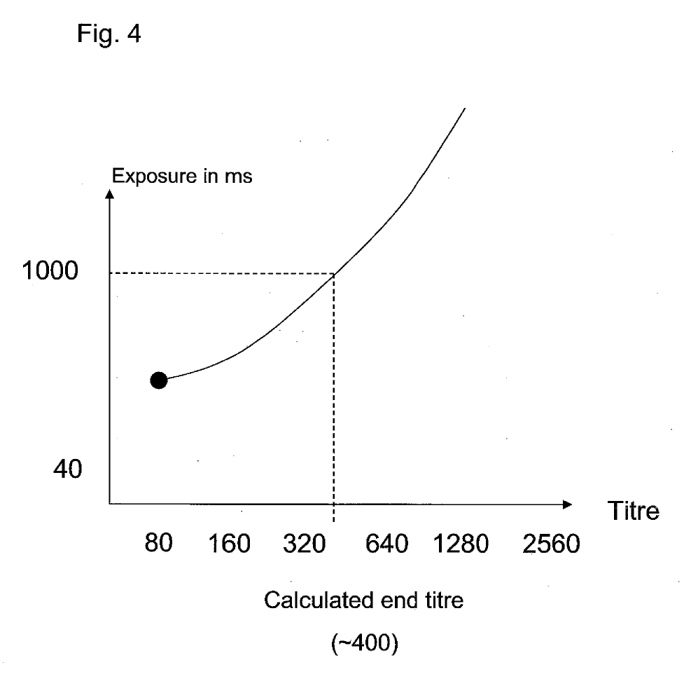

FIG. 4 shows a further measurement of a patient serum with a dot pattern, whereby the end point is unknown. A known dilution titre of 80 was used. The measured exposure time was 200 ms. From this an end tire of ˜400 was calculated by insertion of the values in the calibration function.

The following table 2 provides the measurement results from a second test series (linear calibration function) according again to the invention:

TABLE 2

| Test | | Titre | Titre | | | |
|---|---|---|---|---|---|---|
| row | Serum | Software | Person | Specificity | Deviation | Comment |
| 1. | 2518 | 320 | 640 | dsDNA/SS-A | −1 | |
| | 2520 | 320 | 320 | dsDNA/SS-A | 0 | |
| | 2500 | 640 | 640 | SS-A/SS-B | 0 | |
| | 2507 | 2560 | 5120 | SS-A/SS-B | −1 | |
| | 2519 | 1280 | 1280 | RNP | 0 | |
| | 2522 | 320 | 320 | RNP | 0 | |
| | 2529 | 320 | 640 | Sm | −1 | |
| | 2514 | 1280 | 2560 | RNP | −1 | |
| 2. | 3698 | 1280 | 1280 | Scl-70 | 0 | |
| | 3699 | 640 | AMA 320, | | −4 | No longer linear |
| | | | CENP 10240 | | | at very high |
| | | | | | | titres |
| | 3633 | 2560 | 5120 | Sm/RNP | −1 | |
| | 2423 | 2560 | 10240 | PMScl | −2 | No longer linear |
| | | | | | | at very high |
| | | | | | | titres |
| | 3462 | 1280 | 1280 | SS-A/SS-B | 0 | |
| | 3007 | 2560 | 5120 | dsDNA/SS-A | −1 | |

“Serum” describes a serum provided with an internal reference number.
“Titre Software” describes the titre determined by the software according to the present invention using the proportionality as described above from the known initial titre, the used exposure time and final exposure time.
“Titre Person” describes the determined titre on grounds of the subjective perception of the fluorescence intensity according to the usual procedure to date.
“Deviation” describes a deviation in the subjectively determined titre steps. Thereby a deviation (see above) of +/−1 titre step is not seen as significant and in practise does not play a role in the applied technique.

Hence a surprising result is achieved, that it is possible to achieve a more exact and mistake-free result in determining the end titre using the technique according to the present invention.

The method according to the present invention, the kit according to the invention based on said method, in addition to the test principle are described in detail with the following

a. The Kit according to the present invention contains at least the following contents:
Slides with application sites that are coated with HEp-2 cells, which are optionally sealed with a protective gas,
Wash buffer/sample dilution PBS buffer, pH 7.4±0.1, as a solid substance,
Conjugate comprising anti-human Immunoglobulin (IgG and light chain specific), coupled with FITC,
Covering medium of permanent glycerol solution, phosphate buffered, with anti-fading reagent,
Cover slips,
Positive control; a positive human serum with antibody specificity,
Negative control; a negative human serum,
Measurement system for reading and evaluating the fluorescence patterns

Furthermore, additional common aids are also intended to be included, such as user-defined micropipettes (10, 100, 1000 μl), pipette tips, sample dilution tubes, measurement cylinders or volumetric flasks, a humid incubation chamber, plastic wash bottles and/or staining troughs.

b. The antibodies in diluted patient samples or in control serum react in a first reaction step specifically with the antigens of the HEp2-cells that are fixed to the slide. Unbound components are removed by a wash step after a 30-minute incubation at room temperature. The bound antibodies react in a second reaction step specifically with anti-human antibodies (IgG and light chain specific), which are coupled to fluorescein-isothiocyanate (FITC). Surplus conjugate molecules are then separated from the immunocomplexes bound to the solid phase by a further wash step after a 30-minute incubation at room temperature. Specific fluorescent patterns are observable according to the histological arrangement of antigens in the HEp-2 cells. After covering, the slides are read under a fluorescent microscope (excitation wave length 490 nm, emission wave length 520 nm) with an automated measurement system.

c. In order to extract samples, blood taken from patients by vein puncture is allowed to coagulate and the serum is subsequently isolated by centrifugation. Before application in the assay the sera are brought to room temperature, and optionally briefly shaken in order to ensure an appropriate homogeneity. The patient samples for the assay of the present invention are diluted at a ratio of 1:80 (v/v) with PBS buffer. In addition to this screening dilution, a 1:320 dilution can be applied to safeguard the titre prediction, or for a better evaluation, in case of a potential mixed pattern (EASI recommendation). Starting from the 1:80 (v/v) dilution the samples are further diluted 4-fold in PBS buffer solution, for example 100 μl sample dilution+300 μl PBS buffer.

d. The assay according to the present invention is carried out as follows:
d. 1. The assay reagents are brought to room temperature (RT, 20-25° C.). The slides are removed from their packaging and labelled directly before use in order to avoid contamination.
d. 2. Pipetting of 25 μl of controls (25 μl of the diluted patient serum)
d. 3. Slides are incubated for 30 minutes at room temperature in a humid incubation chamber.
d. 4. The slides are rinsed with PBS solution.
d. 5. The slides are each washed for 2×5 minutes with fresh PBS solution in staining troughs.
d. 6. Slides are singly removed, PBS is allowed to drip off, 1 drop of conjugate is applied to every application site so the application site is completely covered.
d. 7. Slides are incubated for 30 minutes at room temperature in a humid incubation chamber. Slides are protected from direct light.
d. 8. Repeat steps d. 4. and d. 5.
d. 9. Slides are singly removed, PBS is allowed to drip off, a small drop of covering medium is applied to the edge of each application site. The cover slip is then carefully placed on the slide, so that the covering medium forms a bubble-free closed layer.
d. 10. Slide is read by means of the automated system. The underneath surface of the slide should be wiped well!

e. Evaluation of the Results

The automated evaluation system according to the present invention delivers a decision for each application site (positive or negative) in addition to a result regarding the main fluorescence pattern and a recommendation for the end titre (concentration of antibody). Samples that are evaluated by the system as positive can be controlled using saved images on the PC.

A sample is evaluated as ANA negative when the intensity of the fluorescence in the 1:80 dilution is smaller than a predetermined threshold of the software. A sample is evaluated as ANA positive when the intensity of the fluorescence in the 1:80 dilution is greater than a predetermined threshold of the software.

It is a further aspect of the present invention that, for positive ANA results, the software carries out a classification of the fluorescent patterns into the following groups: homogenous, speckled, nucleolar, centromeric, nuclear dots, mitosis, cytoplasm.

Various patterns can be distinguished according to the staining of the cell nucleus of the HEp-2 cells:

e.1. Homogenous: Diffuse staining of the entire cell nucleus, with or without concealing the nucleoli. The pattern can appear speckled in some samples, especially close to the endpoint. The chromosome region of cells in mitosis displays a strong positive fluorescence. Antigens: DNA, histones. Clinical relevance: high titres specific for SLE, lower titres also for Rheumatoid arthritis; histone antibodies are very strongly associated with drug-induced Lupus.
e.2. Peripheral: Smooth staining of the outer areas of the cell nucleus, weaker fluorescence in the inner areas; not all cells of an application site need show this peripheral staining, some cells could exhibit a homogenous pattern. The chromosome region of cells in mitosis shows a strongly positive fluorescence (a thin ring-formed staining with a negative chromosome region of cells in mitosis suggests however antibodies directed against the nuclear membrane). Antigens: DNA, histones. Clinical relevance: high titres in the active phase of SLE, low titres also for other connective tissue disorders.
e.3. Speckled: fluorescent speckles over the entire cell nucleus, very fine to very coarse speckles are possible, depending on the type of antibody. The chromosome region of cells in mitosis normally reacts negatively.
a) Sm and nRNP: coarse speckles, exclusion of the nucleoli, chromosome regions of cells in mitosis is negative. Clinical relevance: Sm antibodies are a highly specific marker for SLE; high anti-nRNP titres are characteristic for MCTD, together with other ANAs in SLE, RA, PSS.
b) SS-A and SS-B: small uniform speckles in an even distribution, chromosome region of cells in mitosis is negative. Clinical relevance: very common in primary Sjögren's Syndrome, less common in SLE, anti-SS-S very common in neonatal Lupus and congenital heart block.
c) Scl-70: fine-density speckles with fluorescence of the nucleoli, the chromosome region of cells in mitosis is positive. Clinical relevance: anti-Scl-70 are effective as a marker for PSS.
d) PCNA: variable fine and coarse speckles in 30-60% of the cells, cells in mitosis can be positive or negative. Clinical relevance: anti-PCNA occur in a small percentage of SLE patients.
   e.4. Centromeric: Discrete speckles over the entire cell nucleus, the number corresponds to the single or multiple chromosome set. The fluorescent pattern of cells in mitosis follows the distribution of chromosomes: pair wise points in the equatorial plane during metaphase, movement apart to the centrosomes during anaphase. A similar pattern (multiple nuclear dots) is caused by NSP-1 (SP100) antibodies, although here the chromosome region of cells in mitosis remains negative. Antigens: centromeric proteins of the chromosomes. Clinical relevance: marker for CREST Syndrome, more seldom for diffuse Scleroderma and Raynaud's phenomenon.
   e.5. Nucleolar: Fluorescence of the nucleoli within the cell nucleus, clearly defined from unstained nuclear plasma. The fluorescence of the nucleoli can be homogenous or speckled (“clumpy”). Often accompanied by a speckled pattern. Antigens: PMScl, RNA Polymerase I, Fibrillin. Clinical relevance: high titres specific for PSS, Polymyositis-Dermatomyositis Overlap, low titres for SLE, Sjögren's Syndrome, Raynaud's Phenomenon.
   e.6. Spindle apparatus: Network of fine threads which connect the centrosomes to one another in cells undergoing mitosis. Antigens: Spindle apparatus of cells in mitosis. Clinical relevance: rare pattern in a number of autoimmune and other disorders (RA, SLE, PBC, Carpal Tunnel Syndrome).
   e.7. Cytoplasm: speckled or thread-like fluorescence in the cytoplasm
a) Ribosomal RNP: finely speckled fluorescence in the entire cytoplasm, often accompanied by a nucleolar pattern (confirmation on other tissue sections is recommended). Clinical relevance: characteristic for some cases of SLE.
b) Jo-1 (PL-7, PL-12): finely speckled with generally weaker fluorescence, mainly in the peri-nuclear region. Clinical relevance: polymyositis, dermatomyositis.
c) Mitochondrial: small uniform speckles in a thread-like arrangement, more dense in the regions near the nucleus (confirmation on other tissue sections is recommended). Clinical relevance: marker for primary biliary cirrhosis (PBC).
d) Cytoskeleton: thread-like, spider web-like fluorescence over the cytoplasm caused by antibodies directed against actin and other components of the cytoskeleton (vimentin, tubulin), confirmation on other tissue sections is recommended. Clinical relevance: several, anti-actin common in autoimmune hepatitis and infectious diseases.

The automated evaluation system delivers a decision for each application site (positive or negative) in addition to a result regarding the main fluorescence pattern and a recommendation for the end titre (concentration of antibody). Samples that are evaluated by the system as positive can be controlled using saved images on the PC.

1. A method for end-titre determination in the determination of antibodies against nuclear and cytoplasmic antigens in human sera by means of an indirect immunofluorescence assay, comprising the following steps
a. reaction and binding of autoantibodies contained within patient sera with and to antigens of HEp-2 cells, leukocytes, Crithidia luciliae and tissue sections which are fixed to a slide,
b. specific fluorescent marking of the bound autoantigens,
c. fluorescent microscopic analysis of the fluorescently marked autoantibodies bound to the slide, in addition to optical recording and evaluation of fluorescent optical images using the fluorescence intensity in an evaluation system,
wherein
d. the evaluation system, before carrying out the method steps, is a) calibrated by means of at least two control sera with defined titre for creating a dilution series, and b) an excitation light of the fluorescence of the system is measured, and
e. the fluorescence intensity of the recorded fluorescent optical images is set in relation to a titre of control sera, thereby providing the end titre of the patient serum to be investigated.

2. The method according to claim 1, whereby the specific fluorescent marking is carried out with fluorescently marked anti-human antibodies.

3. The method according to claim 1, whereby anti-bleaching reagents are added to stabilize the fluorescence signal.

4. The method according to claim 3, whereby the anti-bleaching reagent is 2,3,5,6 tetramethyl-1,4-phenylenediamine.

5. The method according to claim 1, whereby the end titre of the patient serum to be investigated results from a final exposure time, an initial titre of the patient serum and an exposure time of the camera, according to a determined calibration function, which in the simplest linear case can be calculated according to an equation as follows:
$$serum titre = \frac{input titre}{exposure time} * final exposure time$$

6. A device for end titre determination in autoantibody diagnostics in the determination of antibodies against nuclear and cytoplasmic antigens in human sera by means of an indirect immunofluorescence assay, according to the method of claim 1, comprising:
a. a system for image capture by means of a fluorescence microscope with a camera
b. a system for automatic image analysis and determination of captured fluorescence patterns and fluorescence intensities of bound autoantibodies from patient serum, which react with and are bound to antigens of the HEp-2 cells, leukocytes, Crithidia luciliae and tissue sections, which are fixed to the slide.

7. A kit for in vitro diagnostics for the determination of antibodies against nuclear and cytoplasmic antigens in human sera by means of an indirect immunofluorescence assay, comprising at least:
c. a slide with multiple application sites coated with HEp-2 cells for an application of control and patient sera,
d. control sera with different, pre-defined titres for calibration of an optical system of an immunofluorescent microscopic measurement and evaluation system.
e. a fluorescently marked anti-human antibodies for a specific coupling of antibodies that are bound to HEp-2 cells.

8. The kit according to claim 7, whereby a calibration slide is a component of the kit, which exhibits on its surface multiple control sera with defined titres.

9. The kit according to claim 7, whereby the anti-human antibody is anti-human-immunoglobulin.

10. The kit according to claim 9, whereby the anti-human antibody is coupled with fluorescein-isothiocyanate.

11. An executable computer programme, which is saved in a computer-readable-medium comprising a programme code as computer-readable-data, through which the computer is instructed, during active computer operation, to determine an end titre on the basis of a method for end-titre determination in the determination of antibodies against nuclear and cytoplasmic antigens in human sera by means of an indirect immunofluorescence assay according to claim 1.

12. The computer programme according to claim 11, whereby the computer programme is suited for automatic image analysis and determination of captured fluorescent patterns and fluorescent intensities of bound autoantibodies from patient serum, which react with and are bound to antigens of the HEp-2 cells, leukocytes, Crithidia luciliae and tissue sections, which are fixed to the slide.

13. The method according to claim 1, wherein the end titre of the patient serum to be investigated is determined from a final exposure time, an initial titre of the patient serum and an exposure time of the camera.
